(19) United States
(12) Patent Application Publication
(10) Pub. No.: US 2005/0080368 A1
(43) Pub. Date: Apr. 14, 2005

(54) Bandage with cooling capabilities

(76) Inventor: Marni Lynn Hurwitz, Far Hills, NJ (US)

Correspondence Address: SUGHRUE MION, PLLC, 2100 PENNSYLVANIA AVENUE, N.W., SUITE 800, WASHINGTON, DC, 20037, US

(21) Appl. No.: 10/712,020

(22) Filed: Nov. 14, 2003

Related U.S. Application Data
(60) Provisional application No. 60/426,039, filed on Nov. 14, 2002.

(51) Int. Cl. A61F 5/00

(52) U.S. Cl. 602/2

(57) ABSTRACT

A bandage with cooling capabilities includes bandage support members and a cold pack member positioned between the support members. The cold pack member may be a chemical which endothermically reacts with water, positioned adjacent to but separate from a water source inside a common package, to instantly cool the cold pack member upon activation. The cold pack member further comprises a sterile pad member positioned on the bottom side of the bandage, which may include an antibiotic, anesthetic, antipyretic, burn medicament, or combinations thereof.

### FIELD OF THE INVENTION

The present invention relates to a bandage with cooling capabilities, and in particular, a bandage comprising an instant cold pack capable of cooling instantly.

### SUMMARY

In an illustrative, non-limiting implementation, a bandage with cooling capabilities is provided. The bandage comprises bandage support members and a cold pack member positioned between the support members. The cold pack member may comprise a chemical which endothermically reacts with water, positioned adjacent to but separate from a water source inside a common package, to instantly cool the cold pack member upon activation. The cold pack member further comprises a sterile pad member positioned on the bottom side of the cold pack member, which contacts a wound or burn on a user's skin.

### BRIEF DESCRIPTION OF THE DRAWINGS

FIG. 1A is a plan view of the bandage of the present invention;

FIG. 1B is a side-sectional view thereof:

FIG. 2A is a side sectional view of another embodiment of the invention;

FIG. 2B illustrates a package for the liquid reactant; and

FIGS. 3, 4 and 5 are side-sectional views of other embodiments according to the invention.

### DESCRIPTION OF THE PREFERRED EMBODIMENTS

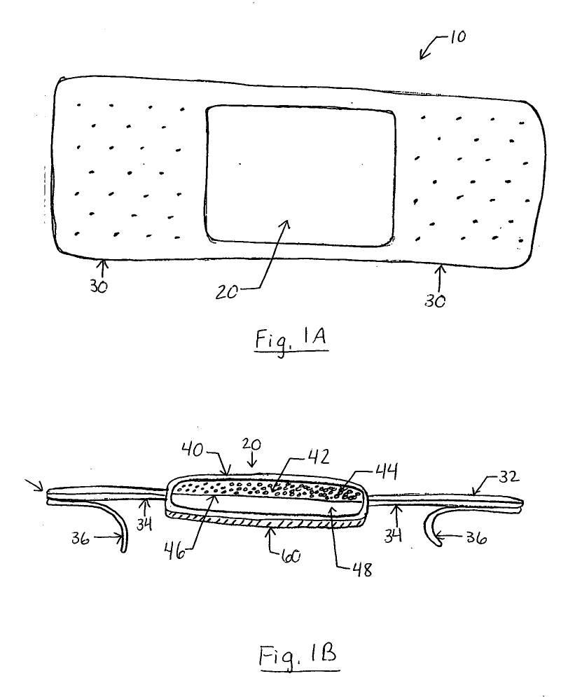

FIGS. 1A and 1B show a bandage with cooling capabilities in accordance with an illustrative non-limiting embodiment of the present invention. As shown in FIG. 1A, the bandage 10 comprises a cold pack member 20 and a bandage support member 30. In this embodiment, cold pack member 20 is an island type member, meaning it is attached to the middle of bandage support member 30. In an alternate embodiment, cold pack member 20 can be integrally attached or sealed to two bandage support members 30, each one attached to either side of cold pack member 20.

A cross sectional view of bandage 10 is shown in FIG. 1B. As shown, bandage support member 30 comprises a top layer 32 and an adhesive bottom layer 34. Adhesive bottom layer 34 further comprises paper-like release members 36, which cover and protect the adhesive on bottom adhesive layer 34 until use. Bandage support member 30 may be made of a flexible, elastic, water resistant, non-absorbing material. Such materials may include woven or non-woven fabrics, a plastic film, a polymeric film, polyurethane, copolyester ether, synthetic rubber, natural rubber or other similar materials. Adhesive bottom layer 34 can be made of commonly known bandage type adhesives, such as, polymeric adhesives including vinyl acetate, acrylic acid, or acrylates.

The non-limiting embodiment of FIG. 1B further shows cold pack member 20 comprising a cold pack container 40. Cold pack container 40 has a first small cavity 42 containing a coolant material 44 in the form of granules, and a second small cavity 48 containing water. Membrane 46 separates cavity 42 from cavity 48. Cold pack member 20 further comprises a pad member 60 laminated to the bottom of cold pack container 40. To activate the cooling capabilities of bandage 10, a user must squeeze, twist or rub, etc. and then shake cold pack member 20. This action will tear or rupture membrane 46 to allow the coolant and water to mix and activate the cooling effect. The user can then place bandage 10 on a wound or burn to provide cooling relief.

Cold pack container 40 is shown as being rectangular in shape, however, it can be shaped like a square, circle, oval, or other suitable shape. Bandage 10 is intended to cover small minor wounds or burns, such as burns by a hot dish, curling iron, etc., therefore, bandage 10 and cold pack member 20 can be sized accordingly. However, bandage 10 and cold pack member 20 can be manufactured in a larger size to accommodate wounds or burns covering a larger area. Bandage 10 may also be used for pain relief associated by aches and pains, such as a headache or muscle strain. In such a use, bandage 10 would provide a hands free method of cooling the head, neck or other body parts by applying and attaching the bandage 10 directly to the skin of the affected area.

Cold pack container 40 needs to be made of material that is both flexible and leak proof. Suitable materials include, but are not limited to, vinyl, polyvinyl chloride, plastic, rubber, vinyl polymer, or a polyester, such as polyethylene. Although not shown, cold pack member 20 can alternatively have a thin insulating layer formed around cold pack container 40 (except for the area opposed to the wound) to increase the duration of the cooling effect.

Coolant material 44 reacts with the water to initiate an endothermic reaction which produces the cooling effect. Coolant material 44, shown in each embodiment, can be in the form of granules, powder or concentrated liquid. The duration of the cooling effect decreases as the size of the granules decreases and is a minimum when the coolant material is in liquid form. Conversely the degree of cooling effect increases with the speed of the endothermic reaction and thus decreases with increasing granule size. Depending on individual needs of manufacturers and suppliers, a proper balance and form of coolant can be found through experimentation. Suitable coolant materials 44 include ammonium nitrate, ammonium sulfamate, ammonium nitrite, sodium carbonate, sodium bicarbonate, sodium nitrate, potassium nitrate, potassium nitrite, urea and methylurea. Preferably, ammonium nitrate is used as coolant 44.

To activate the endothermic reaction, water is used to react with the coolant material 44. Typically, the water is fully or partially encapsulated by a membrane or the like as described herein. However, water can be absorbed into synthetic hydrophillic fibers or other superabsorbant fibers, wherein, limited amounts of water would be released each time a user squeezes cold pack member 20. Therefore, the cooling amount and duration could be partially regulated by the user.

Membrane 46 must be made of a thin material, which is leak proof, yet easily ruptured during use. Suitable polymeric materials include, but are not limited to, polyethylene, polypropylene, polybutylene, polyvinylchloride, polyester, polyethylene terephthalate, vinylidene chloride polymers, and combinations thereof. The membrane 46 can be scored or perforated to facilitate rupturing during use. Membrane 46 can be attached to the sides of cold pack container in the manufacturing stage by heat sealing, ultrasonic or radio frequency welding, adhesive welding or other commonly used technique. Membrane 46 may also be made of candyglass or cellophane with semi-serrations or ridges to break easily.

Pad member 60 is the portion of bandage 10 which contacts the wound or burn. Therefore, pad member 60 must be a sterile, skin friendly material that will not stick to wounds or aggravate burned tissue. Suitable materials may include, but are not limited to, an acryllic, a hydrocolloid, a hydrogel, gauze, cotton, sponges, or a fiber capable of forming a gel on contact with exudate, which can be non-adhering to a wound or burn. Hydrogels may be desirable for use on burns because they promote the cooling effect, may help accelerate healing, provide a cushion between the burn and bandage 10 and generally, a wound exudate does not dry or stick to hydrogels. Pad member 60 may also include various antibiotics and/or anesthetics laminated or applied during the manufacturing process, including, but not limited to, neosporin, camphorated phenol, chloramphenicol, chlortetracycline, erthryomycin, or clyndamycin, as antibiotics and/or xylocaine, lidocaine, benzocaine, butacaine, ethocaine procaine, ethyl aminobenzoate, ethyl chloride, and tetracaine as anesthetics, either alone or in combinations. Further, there may be included common burn relief agents or medicaments and gels such as aloe, glycerin, pregnenolone acetate, pseudocollagen, glycolipids and evening primrose oil, either alone or in combinations, and/or an antipyretic agent.

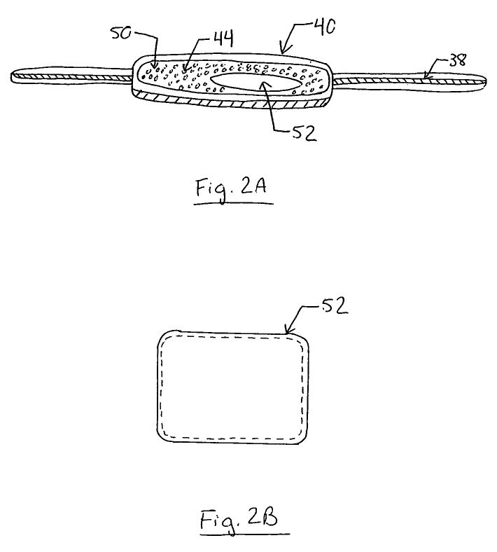

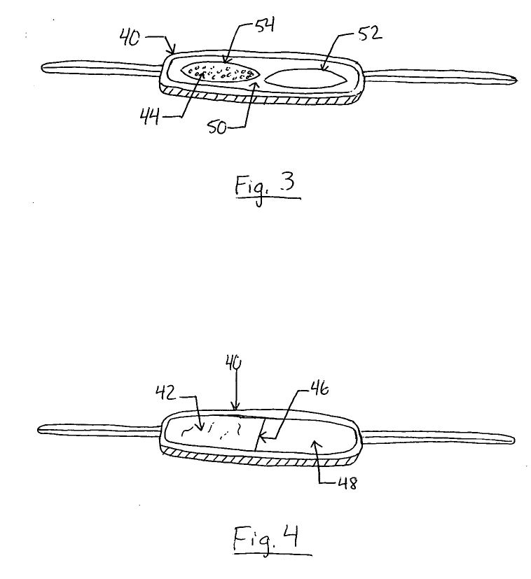

In an alternative embodiment shown in FIGS. 2A and 2B, cold pack container 40 contains one large cavity 50, rather than two small cavities. Coolant 44, shown in the form of granules, is deposited inside cavity 50 along with a water packet 52. Water packet 52, as shown in FIG. 2B, is sealed on all sides so that no water leaks before use. Water packet 52 can be any shape or size, depending on the size of the bandage 10 of which it will be inserted into during manufacturing. In this embodiment, when a user twists or squeezes cold pack member 20, the water packet 52 ruptures. Upon rupturing, the water mixes with the coolant in large cavity 50, producing the cooling effect. Water packet 52 must be made of a material that is leak proof, yet capable of tearing upon use. Suitable polymeric materials include, but are not limited to, polyethylene, polypropylene, polybutylene, polyvinylchloride, polyester, polyethylene terephthalate, vinylidene chloride polymers, and combinations thereof. Water packet 52 can be scored or perforated to facilitate rupturing during use. Water packet 52 can be sealed in the manufacturing stage by heat sealing, ultrasonic or radio frequency welding, adhesive welding or other commonly used technique.

Also shown in FIG. 2A, is an inner support layer 38 positioned between top layer 32 and adhesive bottom layer 34. The inner support layer 38 provides flexible support for the additional weight the cold pack member 20 puts on the bandage 10. Inner support layer 38 can be made of foam or other suitable material.

In another non-limiting embodiment shown in FIG. 3, large cavity 50 contains both a water packet 52 and a coolant packet 54. Coolant packet 54 is shown containing granules of coolant 44, however, the coolant 44 can alternately be in the form of powder or liquid. Both the water packet 52 and coolant packet 54 must be made of a material that is leak proof, yet capable of rupturing during use. Suitable polymeric materials include, but are not limited to, polyethylene, polypropylene, polybutylene, polyvinylchloride, polyester, polyethylene terephthalate, vinylidene chloride polymers, and combinations thereof. Water packet 52 and coolant packet 54 can be scored or perforated to facilitate rupturing during use. Water packet 52 and coolant packet 54 can be sealed in the manufacturing stage by heat sealing, ultrasonic or radio frequency welding, adhesive welding or other commonly used technique.

In an alternative embodiment, coolant packet 54 can be made of a material capable of dissolving in water. Therefore, once water packet 52 is ruptured, the water slowly dissolves coolant packet 54 to release and react with coolant 44. In this embodiment, the cooling effect is extended due to a slower reaction time. Such dissolvable materials may include polyvinyl alcohol, paper, wax paper, clay or clay-like substances. The thicker the packet walls, the slower the mixing of coolant 44 with the water, which will prolong the endothermic reaction. Coolant packet 54 may alternatively be made of a porous material which permits water to gradually flow through the packet, causing the endothermic reaction.

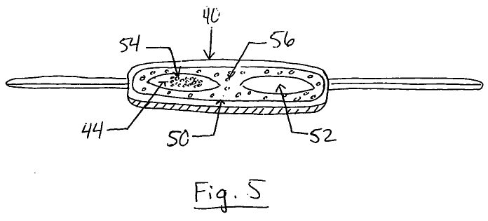

Turning to FIG. 4, cold pack container 40 contains a first small coolant cavity 42, filled with coolant 44 and a second small water cavity 48, similar to FIG. 1B. However, in this embodiment, the membrane 46 runs vertically from the top of cold pack container 40 to the bottom of cold pack container 40. As shown in this embodiment, the coolant 44 is in the form of a liquid. Since the coolant is in liquid form, the reaction will occur immediately upon use, speeding up the cooling process.

In another illustrative non-limiting embodiment of the present invention, FIG. 5 shows large cavity 50 comprising coolant packet 54, which contains coolant 44 and water packet 52. In addition, large cavity 50 contains a second coolant 56. This second coolant 56 can help to prolong the duration of the cooling effect. Preferably, second coolant 56 is urea.

Both water packet 52 and coolant packet 54 can be made of rupturable material, or alternatively, water packet 52 can be made of rupturable material, while coolant packet 54 is made of a dissolvable material. The second coolant 56 may also be placed in a packet. Also, both coolant 44 and second coolant 56 may be in a varied liquid, powder or granule form.

In another non-limiting embodiment, more than two coolants may be used, or a gelling agent such as hydroxypropylmethylcellulose, may be used to prolong the cooling effect.

The previous descriptions of the preferred embodiments is provided to enable a person skilled in the art to make and use the present invention. Moreover, various modifications to those embodiments will be readily apparent to those skilled in the art. It will be appreciated that the above descriptions are intended only to serve as examples, and that many other embodiments are possible within the spirit and the scope of the present invention.

1. A bandage providing a cooling effect, comprising;
at least one chemical agent and at least one solution, which, when mixed, undergo an endothermic reaction;
means for separating said agent and said solution within at least one chamber within a body of said bandage, at least one portion of said separating means being easily broken or ruptured so that said agent and said solution may be mixed;
a skin-adhesive portion for affixing the bandage to the body of a user; and
a sterile portion adapted for contact with the area of the body to be covered by said bandage.

2. A bandage as claimed in claim 1, wherein said sterile portion may be associated with at least one of an antibiotic, an anesthetic, an antipyretic, and a burn medicament.

3. A bandage as claimed in claim 1, wherein said separating means includes one of a frangible, tearable or puncturable member, said chemical agent is provided in dry solid or concentrated solution form, and said solution is water.
